(19) United States
(12) Patent Application Publication
(10) Pub. No.: US 2023/0393223 A1
(43) Pub. Date: Dec. 7, 2023

(54) APPARATUS AND METHOD FOR IMAGING AND CONDUCTING IMAGE-GUIDED PROCEDURES WITH POSITION-SENSING OCULUS

(71) Applicant: Weinberg Medical Physics, Inc., North Bethesda, MD (US)

(72) Inventor: Irving N. WEINBERG, Chevy Chase, MD (US)

(21) Appl. No.: 18/204,744

(22) Filed: Jun. 1, 2023

Related U.S. Application Data
(60) Provisional application No. 63/348,730, filed on Jun. 3, 2022.

(51) Int. Cl. G01R 33/28; A61B 5/055; G01R 33/383

(52) U.S. Cl. CPC G01R 33/287; A61B 5/055; G01R 33/383; G01R 33/283

(57) ABSTRACT

A device and method for imaging includes a magnetic resonance imaging system, and an oculus for inspection of a structure of interest. The oculus includes or is attached to at least one sensor for determining the location of the oculus with respect to the magnetic resonance imaging system or the structure of interest.

### CROSS REFERENCE AND PRIORITY CLAIM

This application claims priority to U.S. Provisional Patent Application Ser. No. 63/348,730, entitled “APPARATUS AND METHOD FOR IMAGING AND CONDUCTING IMAGE-GUIDED PROCEDURES WITH POSITION-SENSING OCULUS” filed Jun. 3, 2022, the entirety of which is incorporated by reference.

### FIELD

Disclosed embodiments are directed to diagnosing and treating diseases in animals, including humans. In particular, disclosed embodiments may be used in medical and veterinary applications. Alternatively, the disclosed embodiments may be used outside of medicine, for example, in the field of inspection.

### BACKGROUND

Surgeons presently use pre-operative imaging studies to plan therapeutic procedures. To help the surgeons, sometimes body and tool landmarks that are visible in the operative field are co-registered with the pre-operative imaging studies, providing the surgeon with feedback as to where the tool is at the time of the procedure. Disclosed embodiments describe the design and use of an oculus and system for use with a magnetic resonance imaging system, which system does not require such landmarks.

### SUMMARY

Disclosed embodiments include an apparatus and method for real-time inspection of a structure-of-interest in a body part including a magnetic resonance imaging system. The apparatus may include at least one sensor capable of determining the location of an inspection device with respect to the magnetic resonance imaging system and/or the structure-of-interest. The inspection device may be an oculus.

In some embodiments, the sensor measures the magnetic field at the location of the inspection device, or oculus, with respect to the MRI and/or structure-of-interest to assist in the determination of the location of the inspection device. In some embodiments, the oculus also includes at least one radio-frequency coil sensitive to radiofrequency signals emitted by structures at or near the location of the inspection device.

### BRIEF DESCRIPTION OF THE FIGURES

Aspects and features of the disclosed embodiments are described in connection with various figures, in which:

FIG. 1 shows an embodiment of the disclosed apparatus having a structure for inspection and/or visualization of a structure of interest;

FIG. 2 shows the apparatus according to the disclosed embodiments as applied to surgery of a human subject; and

FIG. 3 illustrates in flow-chart of a method of performing an image-guided procedure according to the disclosed embodiments.

### DETAILED DESCRIPTION

The present invention will now be described in connection with one or more embodiments. It is intended for the embodiments to be representative of the invention and not limiting of the scope of the invention. The invention is intended to encompass equivalents and variations, as should be appreciated by those skilled in the art.

Disclosed embodiments consist of an apparatus and method for real-time inspection of a structure-of-interest 120 with structure, such as an oculus 100. The apparatus and method are used with or include a magnetic resonance imaging system 140. Said apparatus includes at least one sensor 150 capable of determining the location of oculus 100 with respect to the magnetic resonance imaging (MRI) system 140 and/or the structure-of-interest 120. A structure 100 for inspection and/or visualization of a structure-of-interest (the structure illustrated herein as an “oculus”) may be used by a person carrying out a procedure involving said structure-of-interest. For example, one such person might be a surgeon 110. A procedure may be carried out or planned planned to be carried out in or near a structure-of-interest 120 that may be in an inanimate subject or within or upon a part of a human or animal subject.

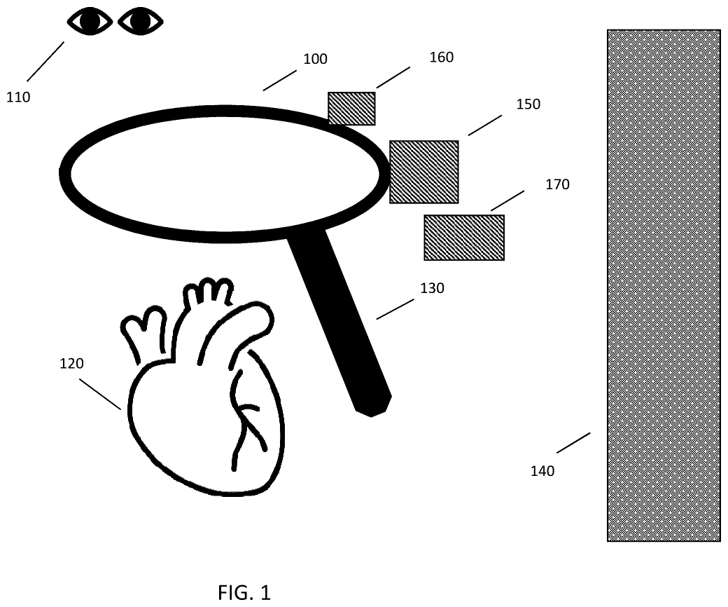

Oculus 100 may have a hollow or at least partially transparent section as in FIG. 1 so that the structure-of-interest 120 is optically visible to the surgeon 110. In an embodiment, oculus 130 may have a screen so that another representation (for example a magnetic resonance image) of the structure-of-interest 120 is displayed on the oculus 130. In an embodiment, another representation (for example a magnetic resonance image) of the structure-of-interest 120 may be displayed on a monitor or other display 170 separate from, and in wireless or wired communication with, or integrated with the oculus.

Oculus 100 may include a device sensitive to electromagnetic energy emitted by the structure of interest 120, such as a radiofrequency coil 160. Oculus 100 may be supported by a handle 130, or other means of affixing or manipulating the oculus in space, which may be handheld. Handle 130 may be affixed to structure-of-interest 120 or body part containing the structure-of-interest or to magnetic resonance imaging system 140 or to some other structure. Position sensor 150 is affixed to or incorporated within oculus 100. Position sensor 150 may be a magnetic sensor, sensitive to magnetic gradients generated by MRI system 140.

It is understood that sensor 150 may be part of the oculus or may be rigidly attached to the oculus (for example on the handle).

It is understood that sensor 150 may be a magnetometer, for example a Hall-type device, or may utilize radiofrequency measurement techniques to determine magnetic field and magnetic field gradient, as is known in the field of MRI as a sniffer or field probe.

It is understood that the oculus operates in a magnetic field produced by the magnetic resonance imaging system 140.

In some embodiments, sensor 150 measures the magnetic field and/or gradient at the location of the inspection device with respect to the MRI and/or structure-of-interest to assist in the determination of the location of the inspection device.

In an embodiment, oculus 130 also includes at least one device sensitive to radiofrequency (“RF”) signals emitted by structures-of-interest and/or nearby structures at or near the location of the oculus 100. Said RF signals may have emanated in response to radiation generated by the MRI system 140, for example as in an MRI pulse sequence. Said sensitive device may be a coil or may be another type of sensor, for example an optically-pulsed magnetometer.

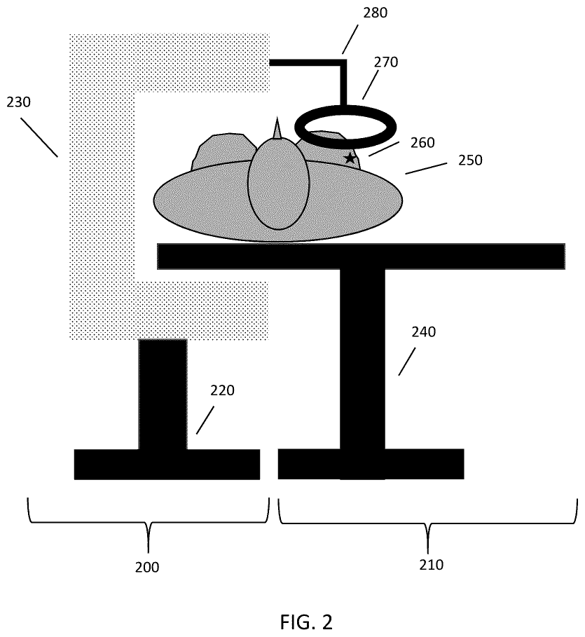

It is understood that oculus 130 may also include a transmitting RF coil. Oculus 130 may be sensitive to radiation emanated by magnetic particles present in structures-of-interest and/or nearby structures, for example as is known in magnetic particle imaging. Oculus 130 may be held by a surgeon or other person using a handle 130 or may be mechanically attached to the patient table or the MRI as shown in FIG. 2.

As illustrated in FIG. 2, an MRI system 200 includes a support structure 220 and array of magnets and electromagnets 230 near a patient structure 210. Patient structure including a support 240 upon which a patient 250 lies. Patient 250 may have a structure of interest 260, for example, a tumor. Oculus 270 is shown near tumor 260, supported by an articulating arm 280. FIG. 2 is similar to, and incorporates by reference, the intra-operative MRI described in U.S. patent application Ser. No. 18/078,661 entitled “APPARATUS AND METHOD FOR C-ARM MRI WITH ELECTROPERMANENT MAGNETS”.

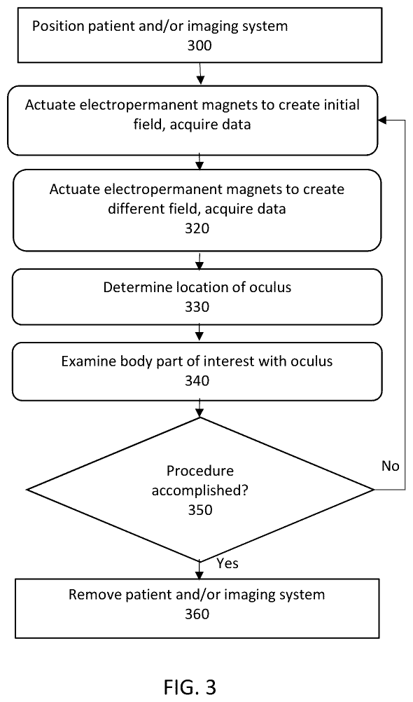

In FIG. 3, a method begins with positioning of the subject (“patient”) and/or magnetic resonance imaging system 300 so that at least one structure-of-interest of the subject is exposed for access during an anticipated procedure to accomplish certain goals (e.g., surgical excision of a tumor). Electropermanent magnets are actuated to create initial magnetic field configuration, and data are collected to assist in forming a magnetic resonance image 310. Electropermanent magnets may be actuated to create another magnetic field configuration, and data are collected to assist in forming a magnetic resonance image 320. The location of oculus is determined 330. For example, in some embodiments, the location of the oculus may be determined by sensing magnetic fields produced by magnet array of the MRI. An image of the structure-of-interest and possibly surrounding structures in a patient may be optionally collected with the oculus 340. In some embodiments, the oculus contains a radio-frequency coil to collect radiofrequency signals emanating from the body-part-of-interest. A decision is made by the operator as to whether the goals of the procedure have been accomplished 350. If the goals have been accomplished, then the patient and/or imaging system is removed 360. Otherwise some or all of the previous steps are repeated. It is understood that the operations of FIG. 3 may be carried out sequentially or may be carried out simultaneously or in different orders.

It is understood that the proximity of oculus to the structure-of-interest may result in improved collection of signals emanating from structure-of-interest and hence may result in improved image quality. It is understood that the distance from the oculus to the structure of interest may be less than one meter, or less than 15 centimeters.

It is understood that within this disclosure, the terms “structure-of-interest”, “structure of interest” are used interchangeably, and that the commonly-used terms “region of interest” and “volume of interest” could refer (respectively) to the region or volume possibly containing a structure-of-interest.

It is understood that MRI pulse sequences used with the invention may include methods of viewing structures-of-interest in depth, for example displaying multiple slices (each at a different depth) on a screen or providing a maximum intensity image.

It is understood that images obtained prior to use of the oculus may be combined with images obtained using the oculus. It is understood that images obtained with the oculus may be combined with images obtained by the MRI without using the oculus, for example by putting the oculus image of the structure of interest as an inset within an image of a larger region of the subject.

It is understood that if the MRI utilizes electropermanent magnets, the static field strength may change in order to highlight regions of increased cellularity, as disclosed in the U.S. Ser. No. 17/987,272 by Weinberg entitled “Method for intrinsic contrast MRI with electropermanent magnets”, and patent application publication US 2017/0227617 by Irving Weinberg, entitled “Method and apparatus for manipulating electropermanent magnets for magnetic resonance imaging and image guided therapy”, incorporated herein by reference.

It is understood that various pulse sequences or extrinsic contrast administrations may be utilized by the MRI to highlight features in the structure of interest, for example a T2-weighted image.

It is understood that if the MRI utilizes electropermanent magnets, the static field strength may change in order to pre-polarize protons or electrons in the structure-of-interest, as disclosed in the U.S. Pat. No. 9,411,030 by Weinberg entitled “Apparatus and method for decreasing bio-effects of magnetic gradient field gradients”, incorporated herein by reference.

It is understood that the MRI may utilize electropermanent magnets to implement the change of static field.

It is understood that the magnetic field generated by the MRI may extend beyond the physical limits of the MRI and/or beyond the volume enclosed by the MRI components, as disclosed in U.S. patent application Ser. No. 18/078,661 by Weinberg in “Apparatus and method for C-arm MRI with electropermanent magnets”.

In an embodiment, oculus 270 contains one or more radio-frequency coils (e.g. 160 in FIG. 1) to transmit and/or receive collect radiofrequency signals emanating from the body-part-of-interest.

In an embodiment, oculus 270 contains one or more sensors sensitive to radiofrequency signals emanating from the body-part-of-interest, in which said sensors are not coils. For example, the sensor may be an optically-pumped magnetometer or other quantum measurement device.

It is understood that a computer and power supplies and other electronic components (not shown in the figures) may be required to coordinate, create collect, calculate, and display data from the apparatus and carry out the method of the invention.

Those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments but should instead be defined only in accordance with the following claims and their equivalents.

1. An apparatus comprising:
a magnetic resonance imaging system, and
an oculus for inspection of a structure of interest,
wherein the oculus includes or is attached to at least one sensor configured to determine the location of the oculus with respect to the magnetic resonance imaging system or the structure of interest.

2. The apparatus of claim 1, wherein the at least one sensor is sensitive to electromagnetic fields emanating from the structure of interest.

3. The apparatus of claim 2, wherein the sensor is a coil.

4. The apparatus of claim 1, wherein the oculus includes a transmitter of electromagnetic fields absorbed by the structure of interest.

5. The apparatus of claim 4, wherein the transmitter is a coil.

6. The apparatus of claim 1, wherein a portion of the oculus is hollow or at least partially transparent.

7. The apparatus of claim 1, wherein a portion of the oculus is configured to display an image of the structure of interest.

8. The apparatus of claim 1, wherein the structure of interest is not enclosed by a volume of the magnetic resonance imaging system.

9. The apparatus of claim 1, wherein the magnetic resonance imaging system includes one or more electropermanent magnets.

10. A method of inspecting a structure of interest comprising:
providing a magnetic resonance imaging system, and
positioning an oculus including or is attached to at least one sensor configured to determine the location of the oculus with respect to the magnetic resonance imaging system or the structure of interest in the proximity of a structure of interest, and operating the oculus in the presence of a magnetic field produced by a the magnetic resonance imaging system.

11. The method of claim 10 wherein a procedure is guided based in images produced with the aid of the oculus.

12. The method of claim 10, wherein the at least one sensor is sensitive to electromagnetic fields emanating from the structure of interest.

13. The method of claim 12, wherein the sensor is a coil.

14. The method of claim 10, wherein the oculus includes a transmitter of electromagnetic fields absorbed by the structure of interest.

15. The method of claim 14, wherein the transmitter is a coil.

16. The method of claim 10, wherein a portion of the oculus is hollow or at least partially transparent.

17. The method of claim 10, wherein a portion of the oculus is configured to display an image of the structure of interest.

18. The method of claim 10, wherein the structure of interest is not enclosed by a volume of the magnetic resonance imaging system.

19. The method of claim 10, wherein the magnetic resonance imaging system includes one or more electropermanent magnets.
